(19) United States
(12) Patent Application Publication
(10) Pub. No.: US 2015/0157188 A1
(43) Pub. Date: Jun. 11, 2015

(54) SYSTEMS AND APPARATUSES FOR IMPROVED VISUALIZATION OF ENDOSCOPIC IMAGES

(71) Applicants: Ahmnon D. MOSKOWITZ, Rockville, MD (US); Mosheh T. MOSKOWITZ, Rockville, MD (US); Nathan C. MOSKOWITZ, Rockville, MD (US)

(72) Inventors: Ahmnon D. MOSKOWITZ, Rockville, MD (US); Mosheh T. MOSKOWITZ, Rockville, MD (US); Nathan C. MOSKOWITZ, Rockville, MD (US)

(21) Appl. No.: 14/460,330

(22) Filed: Aug. 14, 2014

Related U.S. Application Data
(60) Provisional application No. 61/866,038, filed on Aug. 14, 2013.

(51) Int. Cl. A61B 1/00; A61B 1/018; A61B 1/06; A61B 1/04

(52) U.S. Cl. CPC A61B 1/00048; A61B 1/04; A61B 1/018; A61B 1/06; A61B 1/00009; A61B 1/0011

(57) ABSTRACT

An endoscope video imaging subsystem including an image sensor, a plurality of image forming optical elements, a video processor, a video processor display signal output interface, an illumination subsystem with one or more light sources, and at least one light transfer and projection element. The endoscope is in communication and sends images through an electronic interface to a head-mounted eyepiece configured to be worn by a user, wherein the eyepiece includes an optical assembly combined with displayed content, an integrated processor for handling content for display to the user, and an integrated image source for introducing the content from the camera to the optical assembly.

### CROSS-REFERENCE TO RELATED APPLICATIONS

This application claims the benefit under Title 35, U.S.C. §119 (e) of U.S. provisional application 61/866,038, filed on Aug. 14, 2013, the entire contents of which are hereby incorporated by reference in their entirety and for which priority is claimed under 35 U.S.C. §120.

### FIELD OF THE INVENTION

The present invention generally relates to the field of endoscopy and endoscopic surgery, and more particularly, to endoscopic viewing apparatuses for endoscopy and endoscopic surgery, and more particularly, to a moveable screen holder system for an endoscope.

### BACKGROUND OF THE INVENTION

Endoscopy allows a visualization device (an endoscope) that allows the surgeon to see what is happening inside the patient. There can be additional surgical tools the surgeon manipulates to perform the desired procedure.

More recently, endoscopic surgical tools have been developed in which the endoscopic function and the surgical function are combined into a single device. There are two particular surgical advantages of this combined function device: 1) only a single device needs to be inserted into the patient and manipulated by the surgeon and 2) the visualization system in the endoscope is pre-aligned in the direction of the effector end of the surgical tool.

There currently exist a variety of conventional head mounted displays. Some contain an optical mixer which is made of partly reflecting surfaces. For example, U.S. Pat. Nos. 6,829,095 and 7,724,441 teach this capability for projected images as well as simultaneously viewing the ambient environment.

### SUMMARY OF THE INVENTION

The present invention recognizes that there is a need for a surgical apparatus and method that will allow the surgeon to see ambient view of the image while simultaneously viewing the area of the procedure so there will be an improvement of hand eye coordination and reduce the risk of surgical error. A variety of conventional moveable devices such as lamps and other devices have been provided that can make the handling of surgical equipment easier. For example, U.S. Pat. Nos. 870,429, 3,858,578, and 5,513,827, teach examples of elements of such moveable devices. U.S. Pat. No. 8,253,692 and U.S. application Ser. No. 13/296,795 teach exams of a transparent screen display.

An optical head-mounted display (OHMD) can be in the form of goggles, spectacles, or another type of head gear video display unit. An optical head-mounted display (also known as Smart glasses) uses an optical mixer which is made of partly silvered mirrors. It has the capability of reflecting projected images as well as allowing the user to look through it. Many companies are developing head mounted displays, such as Microsoft, Google, Olympus, Sony, Osterhout, Epson, and Fujitsu. An example of a head mounted augmented reality system is illustrated in U.S. Pat. No. 8,313,192.

Various head mounted technologies have been developed either using curved mirror based technology or waveguide based technology which can be broken down into further subcategories: diffraction optics, holographic optics, polarized optics, reflective optics, and technology that are switchable amongst the aforementioned optics.

In most endoscopic surgical procedures, the surgeon typically uses an endoscope which is in communication with a display device such as a distant television monitor for observation.

The present invention recognizes that one drawback is that when a surgeon looks at the screen, he/she cannot simultaneously view the ambient view when reassessing positional and tool changes.

The present invention recognizes that there is a need for a truly integrated by design endoscopic system that makes it easier for a surgeon to both see the ambient view of the patient while simultaneously performing the medical procedure in order to maximize surgical efficiency and ergonomics, and to improve surgical technique and diminish risks.

The present invention solves these and other problems by providing an apparatus and tool or device for viewing and performing endoscopic procedures.

An exemplary embodiment of the invention comprises a video endoscope that is in electronic communication with a head piece, wherein a medical worker views the real ambience proximate to the incision sight as well as the augmented frame stream on a semi-transparent display module streamed from the endoscope. It will be appreciated that during the procedure a physician can give equal attention to both the area proximate to the endoscope camera as well as the view proximate to the incision sight.

In an alternative exemplary embodiment of the invention, the glasses can be folded into a device fixed to a user's ear, like a Bluetooth, and expand to move along the temple, and forehead upon a ‘start use’ request. Additionally, the holography could be minimized sufficiently for the device to house a projective system to project the image in front of the user's eyes.

The exemplary device comprises a video imaging subsystem where generally there would be a video image sensor and compatible support optics and electronics whereby a video image signal stream of the region immediately in front of the endoscopic device is produced and is in electronic communication with the viewing device. In yet another exemplary embodiment of the invention, a device comprises an illumination subsystem; the subsystem comprising light sources and light transfer and projection optical elements, whereby illumination for the video imaging subsystem is projected on the viewing apparatus.

The glasses also known in the art as ‘optical head mounted display’ are in electronic communication with the endoscope either remotely or with wires. This will allow the simultaneous viewing of the endoscopic images as well ambient view of the incision area. In other exemplary embodiments, the endoscope in addition to sending messages to the glasses can transmit images to another display unit.

There is a microprocessor connected with the imaging module through a uni-directional bus, wherein the microprocessor receives the captured images from the imaging module, wherein the microprocessor combines the captured images and form a continuous or discrete frame stream. In an embodiment, the display module is connected to the microprocessor through a uni-directional bus, wherein the display module augments the combined images over a display screen, wherein it is viewed on at least one lens, or in another embodiment, viewed on both lenses. These features can enable a medical professional to simultaneously view the ambience proximate to the incision sight as well as the augmented frame stream due to a semi-transparent nature of the display module.

The exemplary embodiments can be configured to be in electronic communication with a flat screen monitor. The monitor is connected to an apparatus that allows for rotational movement of the flat screen to where the said screen can be within general proximity of the surgeons view so as to be able to see the ambient view as well as the images.

The exemplary embodiments can be configured to be easily moveable so as to provide manipulation by the user. The manipulative elements comprise, for example, one or more linkages, ball and socket joints, moveable joints, and/or an articulating arm or arms, or a gooseneck (e.g., a manipulatable, bendable neck that retains its position). The screen can also be held by hinge support so it can easily be attached and detached. The exemplary embodiments also can include a cover. The cover can be autoclaveable or disposable. There can be a cover for the screen and/or a cover for the linkages that are part of the apparatus.

In one exemplary embodiment, the screen is an LCD. In a further exemplary embodiment, the screen is semi-transparent such that the images can be augmented over the body of a patient wherein a medical professional views a real ambience as well as the augmented frame. This can be done with an OLED, which allows light to pass in multiple directions. One such example is illustrated in U.S. Pat. No. 8,212,744, the contents of which are incorporated herein by reference in their entirety. Having a transparent screen can allow both the doctor and the assistants to view the images and provide the maximum amount of hand eye coordination of a physician hence substantially reducing the possibility of error and maximizing the likelihood of success of a medical procedure.

An exemplary endoscope device comprises an extended, multi-channeled, tubular body and a multi-functional handle assembly wherein the tubular body may be inserted through an opening into a patient's body while the handle assembly remains exterior to the body.

In another exemplary embodiment of the invention, the endoscope further comprises a video imaging subsystem, for example, comprising a video image sensor and compatible support optics and electronics whereby a video image signal stream of the region immediately in front of the device is produced. In yet another exemplary embodiment of the invention, the device comprises an illumination subsystem, the subsystem comprising light sources and light transfer and projection optical elements, whereby illumination for the video imaging subsystem is projected on the region the ambient view of the patient.

The screen provided may have a size, for example, of no more than 20 inches in a direction, so as to avoid obstructing the area proximate to the incision area. If needed, the screen can be made smaller (e.g., 16-65 square inches of surface area). The screen can be a mid-sized screen (e.g., 65-100 sq. inches, 100-125 sq. inches, 125-150 sq. inches, 150-175 sq. inches, or 175-200 sq. inches). The screen can be over 200 square inches. However, the present invention recognizes that a screen over 200 sq. surface inches would be fine if the screen is maintained or positioned in a generally vertical position throughout the medical procedure; however, such a screen size may reduce the viewing area proximate the incision area, and therefore, may be less advantageous when positioned in the horizontal position.

The position of the screen in relation to the medical professional's line of sight and the incision/opening area is important. The screen would generally be in an intermediate area in-between the incision area and the medical professional's field of vision. The screen can also be moved to a position that is either generally proximate or substantially proximate to the surgeon's field of vision. The perspective distances in the aforementioned examples can be, for example, 1-6 inches, 6-12 inches, 12-18 inches, 18-24 inches, or 24-30 inches. In an exemplary embodiment of the invention, it is advantageous to have the monitor positioned within the intermediate area in-between the incision sight and the physician's field of vision. The advantage of this is that a surgeon can position the monitor to wherever they choose in a horizontal and/or vertical direction, and a doctor can move the screen to a location that is lateral, medial, or distal to either the instrument or the incision sight.

Other features and advantages of the present invention will become apparent to those skilled in the art upon review of the following detailed description and drawings.

### BRIEF DESCRIPTION OF THE SEVERAL VIEWS OF THE DRAWINGS

These and other aspects and features of embodiments of the present invention will be better understood after a reading of the following detailed description, together with the attached drawings, wherein:

FIG. 1 illustrates a moveable screen holder and endoscope system according to an exemplary embodiment of the invention;

FIG. 2A illustrates an exemplary embodiment having a generally rigid support;

FIG. 2B illustrates an exemplary embodiment having a combination of a rigid support and a gooseneck movement at the distal end;

FIG. 2C illustrates an exemplary embodiment having a gooseneck is at the center;

FIG. 2D illustrates an exemplary embodiment in which a distal portion is made of a gooseneck mount;

FIG. 2E illustrates an exemplary embodiment having a support where movement is entirely for gooseneck movement;

FIG. 2F illustrates an exemplary embodiment wherein movement is in part by ball and joint movement;

FIG. 2G illustrates an exemplary embodiment wherein a screen is held by a hinge support and rotated to a desired position by a ball and socket;

FIG. 3 illustrates an enlargement of a screen holder according to an exemplary embodiment of the invention;

FIG. 4A illustrates an exemplary embodiment in which a screen is attached to a stand with wheels;

FIG. 4B illustrates an exemplary embodiment in which a screen is attached to a ceiling mount;

FIG. 4C illustrates a plurality of screens that can be used for a surgical technique according to an exemplary embodiment of the invention;

FIG. 4D illustrates a view of a surgeon using the exemplary embodiment shown in FIG. 4A;

FIGS. 5A and 5B schematically illustrate a surgical endoscopic according to an exemplary embodiment of the invention;

FIG. 5C is a partial, perspective view of a surgical endoscopic according to an exemplary embodiment of the invention;

FIG. 6A illustrates an endoscope and augmented reality system according to an exemplary embodiment of the invention;

FIG. 6B illustrates a view of a surgeon using an optical head mount according to an exemplary embodiment of the invention;

FIG. 7A illustrates a block diagram of a wireless endoscopic system according to an exemplary embodiment of the invention;

FIG. 7B illustrates a block diagram of a wired endoscopic system according to an exemplary embodiment of the invention;

FIG. 7C schematically illustrates wireless communication between the endoscope and viewing apparatus according to an exemplary embodiment of the invention;

FIGS. 7D(i) and 7D(ii) illustrate wireless communication systems between the endoscope and viewing apparatus according to exemplary embodiments of the invention;

FIG. 8: An exemplary embodiment where the screen is attached to the device by a gooseneck according to an exemplary embodiment of the invention.

The drawings are not necessarily drawn to scale. Emphasis has instead been placed upon illustrating the principles of the invention.

### DETAILED DESCRIPTION OF THE EXEMPLARY EMBODIMENTS OF THE INVENTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

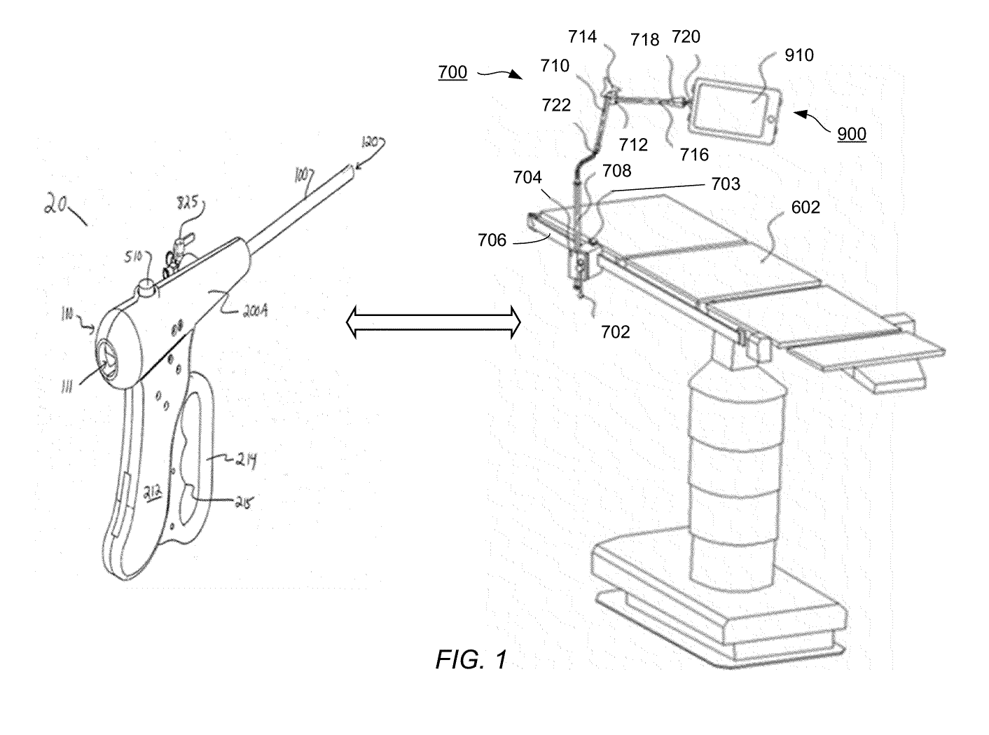

FIG. 1 is an exemplary embodiment of a moveable screen holder 700 that is designed to be in electronic communication with an endoscope 20.

With reference to FIG. 1, an exemplary endoscope 20 will be generally described. Examples of an endoscope 20 will be described in greater detail below. The endoscope 20 has a distal end 120 and a proximal end 110. The endoscope 20 includes a control handle assembly having an exterior housing 200A. A tubular body assembly 100 comprises a thin walled, hollow, typically metal tube extending from a distal end 120 to the distal tip of the control handle assembly 200A. In this exemplary embodiment, the control handle assembly 200 has an exterior housing 200A that comprises a pistol style handle grip 210 where reaction grip 212 is fixed and integral to housing 200A whilst articulated grip 214 is D-shaped with a number of finger indentations 215. On the proximal side of handle assembly 200A, fluid I/O ports, with a three position control valve 825 providing off, irrigate, or suction, is disposed above handle grip 210. While convenient for left handed valve operation (when surgical tool is held in the right hand) this valve and port may be disposed on the right side as a surgeon's preference item.

A display output interface 510 is disposed on the top of housing 200A. In this exemplary embodiment, it is assumed that the display signal will be delivered to the video display though this interface. The specific electrical connector configuration in output interface 510 may be selected for the particular application and can depend, for example, at least in part, on what level of video signal processing is performed on-board the surgical tool and how much is off-loaded to a remote video processing unit. In some exemplary embodiments, output interface 510 may be an analog interface such as S-video, for example, while in other embodiments it may conform to a digital video standard. In other embodiments, wherein the display signal is delivered to the video display wirelessly, the wireless version of display signal output interface 510 may be internal to housing 200A and would not therefore be visible in this view. In yet another variation, a wireless transmitter module may be connected to display signal output interface 510 to provide wired or wireless display communication capabilities with the same system embodiment.

The exemplary embodiment illustrated in the Figures, it should be noted, is a baseline configuration in which certain features have been moved to remote modules. For example, the exemplary embodiment does not contain on on-board power supply, that is, a battery. In other embodiments, a battery compartment is built into housing 200A, typically in reaction grip 212. In this embodiment, power for the video camera and illumination subsystem is brought in, typically, through interface 510.

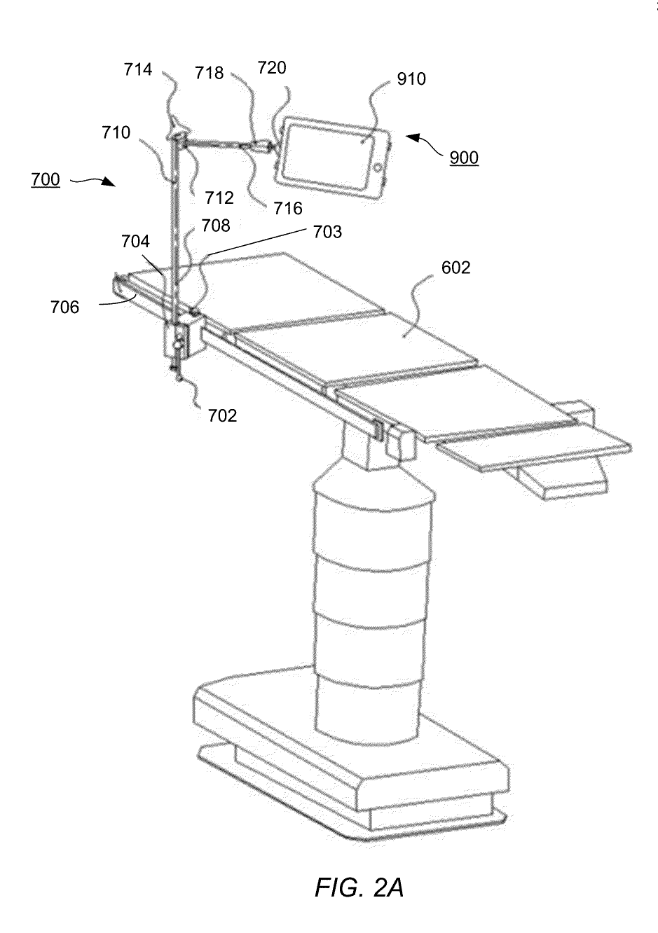

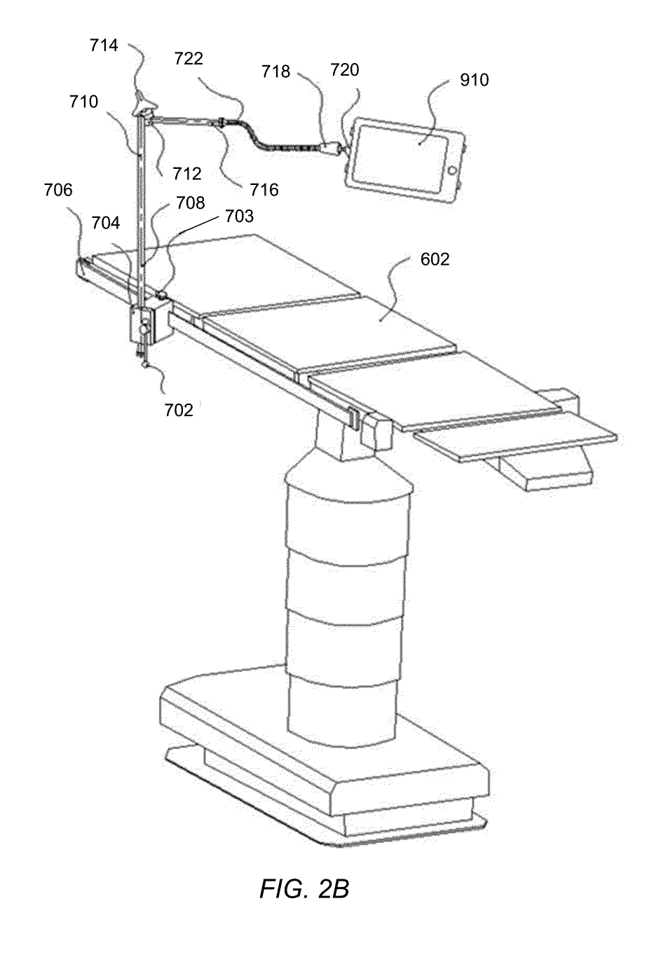

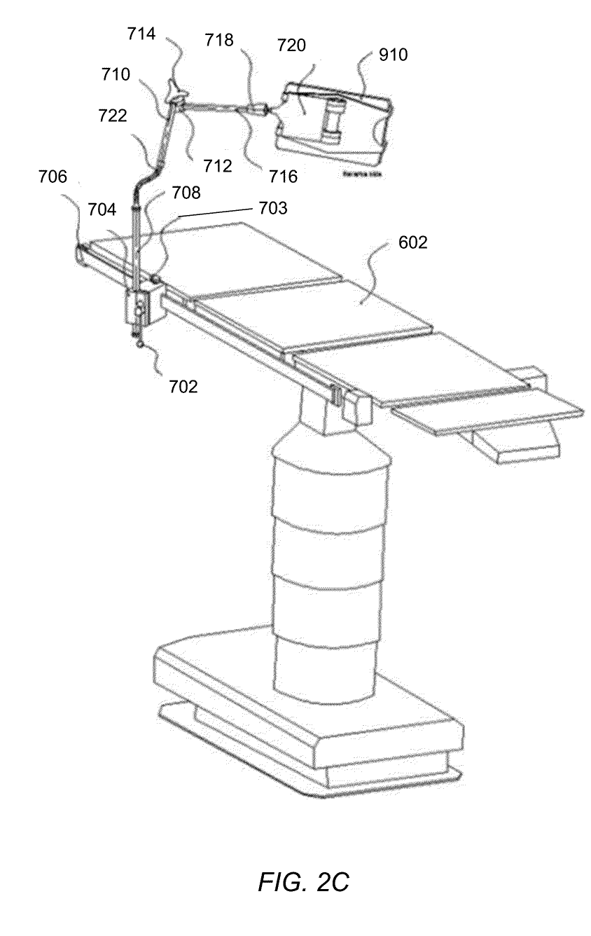

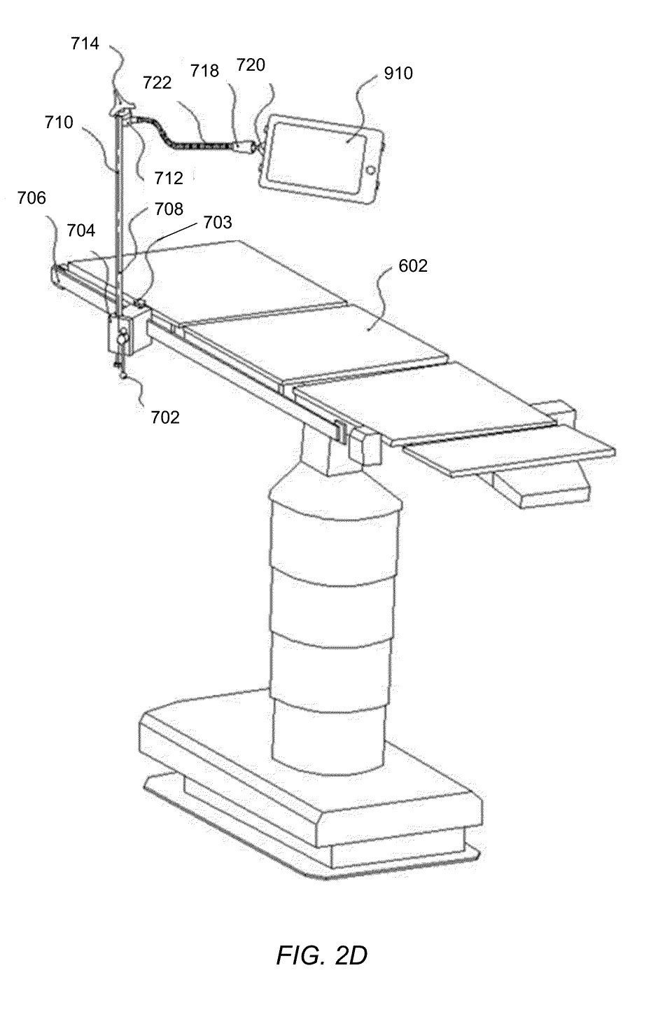

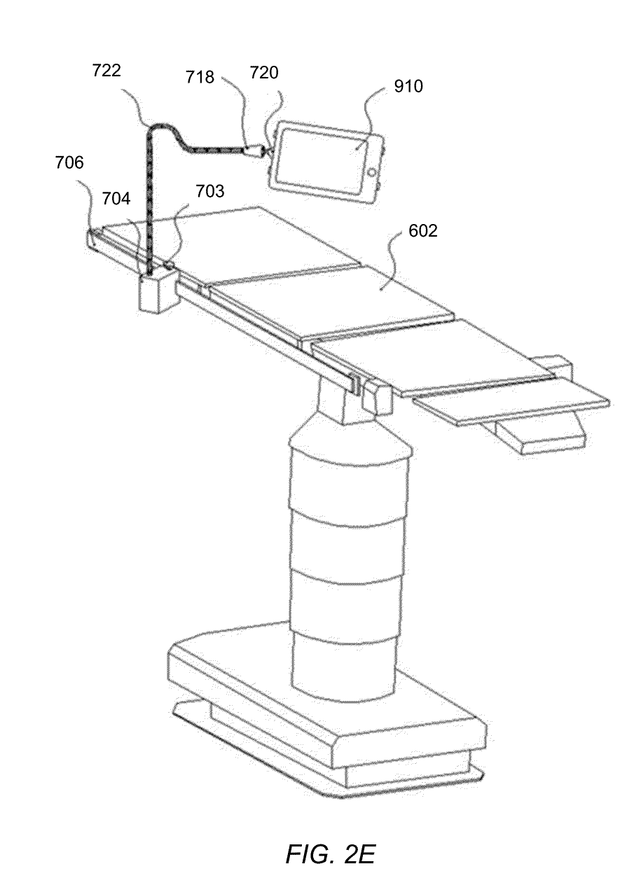

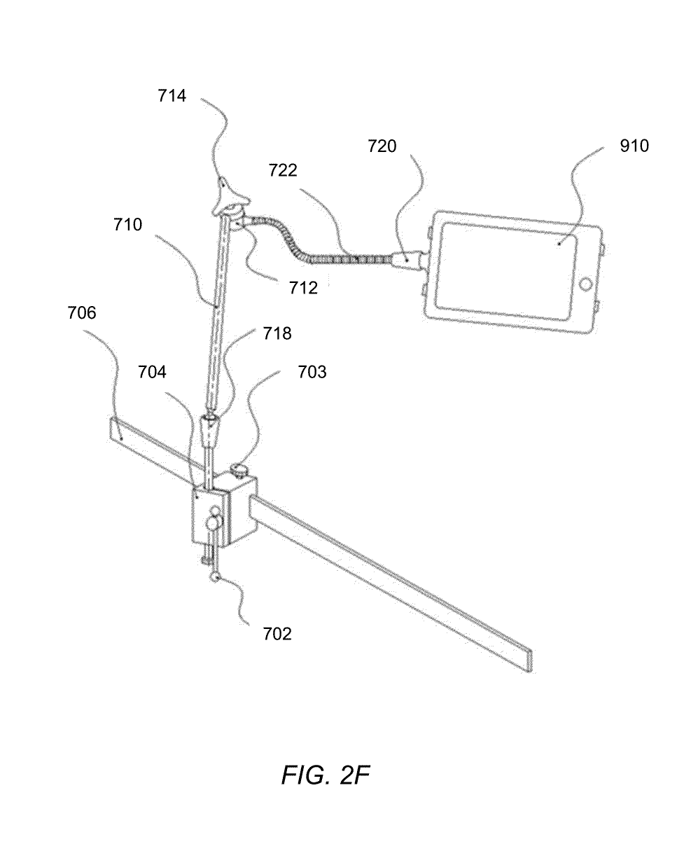

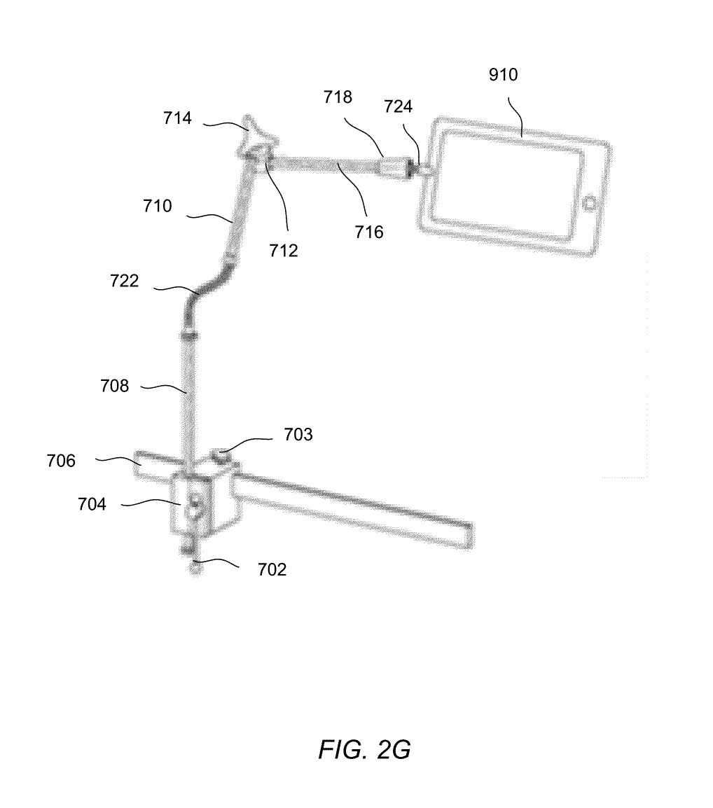

With reference again to FIG. 1, a moveable screen holder system 700 is designed to be in electronic communication with an endoscope 20. The features of the moveable screen holder system 700 will be described in greater detail with reference to the exemplary embodiments illustrated in FIGS. 2A-8. The moveable screen holder system 700 allows the screen 900 to be placed in a position desired by the surgeon. In this embodiment, the system 700 can be configured to be easily attached and detached from a table 602. In other exemplary embodiments, the system 700 can be configured to be separate from the table 602 such as a moveable stand or holder (described in greater detail below).

FIGS. 2A, 2B, 2C, 2D, 2E, and 2F are alternative exemplary embodiments of a moveable screen holder system 700 for endoscopic surgery.

With reference to FIG. 2A, the moveable screen holder system 700 includes a handle 702, a support 704, a flat ruler 706. A screw 703 can be provided for securing a position of the support 704 on the flat ruler 706. The system 700 includes a round ruler 708, a second lever 710, a first lever 716, and a swivel joint 712, with a fixing handle 714, coupling the second lever 710 to the first lever 716. The system 700 includes a ball joint 718 coupling the first lever 716 to a screen holder 720 configured to hold and secure a screen 910 of a display device 900. The system 700 can be coupled to a table 602.

With reference to FIG. 2B, a moveable screen holder system 700 includes a handle 702, a support 704, a flat ruler 706. A screw 703 can be provided for securing a position of the support 704 on the flat ruler 706. The system 700 includes a round ruler 708, a second lever 710, a first lever 716, and a swivel joint 712, with a fixing handle 714, coupling the second lever 710 to the first lever 716. The system 700 includes a gooseneck 722 coupling the first lever 716 to a ball joint 718, which in turn is coupled to a screen holder 720 configured to hold and secure a screen 910 of a display device 900. The system 700 can be coupled to a table 602.

With reference to FIG. 2C, a moveable screen holder system 700 includes a handle 702, a support 704, a flat ruler 706. A screw 703 can be provided for securing a position of the support 704 on the flat ruler 706. The system 700 includes a round ruler 708, a second lever 710, a first lever 716, and a swivel joint 712, with a fixing handle 714, coupling the second lever 710 to the first lever 716. The system 700 includes a gooseneck 722 coupling round ruler 708 to the second lever 710. The system 700 includes a ball joint 718 coupling the first lever 716 to a screen holder 720 configured to hold and secure a screen 910 of a display device 900. The system 700 can be coupled to a table 602.

With reference to FIG. 2D, a moveable screen holder system 700 includes a handle 702, a support 704, a flat ruler 706. A screw 703 can be provided for securing a position of the support 704 on the flat ruler 706. The system 700 includes a round ruler 708, a second lever 710, and a swivel joint 712, with a fixing handle 714. The system 700 includes a gooseneck 722 coupling the swivel joint 712 to a ball joint 718, which in turn is coupled to a screen holder 720 configured to hold and secure a screen 910 of a display device 900. The system 700 can be coupled to a table 602.

With reference to FIG. 2E, a moveable screen holder system 700 includes a support 704, a flat ruler 706. A screw 703 can be provided for securing a position of the support 704 on the flat ruler 706. The system 700 includes a gooseneck 722 coupling the support 704 to a ball joint 718, which in turn is coupled to a screen holder 720 configured to hold and secure a screen 910 of a display device 900. The system 700 can be coupled to a table 602.

With reference to FIG. 2F, a moveable screen holder system 700 includes a handle 702, a support 704, a flat ruler 706. A screw 703 can be provided for securing a position of the support 704 on the flat ruler 706. The system 700 includes a ball joint 718 coupling the support 704 to a second lever 710. A swivel joint 712, with a fixing handle 714, couples the second lever 710 to a gooseneck 722, which in turn is coupled to a screen holder 720 configured to hold and secure a screen 910 of a display device 900. The system 700 can be coupled to a table 602.

With reference to FIG. 2G, a moveable screen holder system 700 includes a handle 702, a support 704, and a flat ruler 706. A screw 703 can be provided for securing a position of the support 704 on the flat ruler 706. The system 700 includes a round ruler 708, a second lever 710, a first lever 716, and a swivel joint 712, with a fixing handle 714, coupling the second lever 710 to the first lever 716. The system 700 includes a gooseneck 722 coupling round ruler 708 to the second lever 710. The system 700 includes a ball joint 718 coupling the first lever 716 to a hinge support 724 on the screen holder 720, which is configured to hold and secure a screen 910 of a display device 900. The system 700 can be coupled to a table 602.

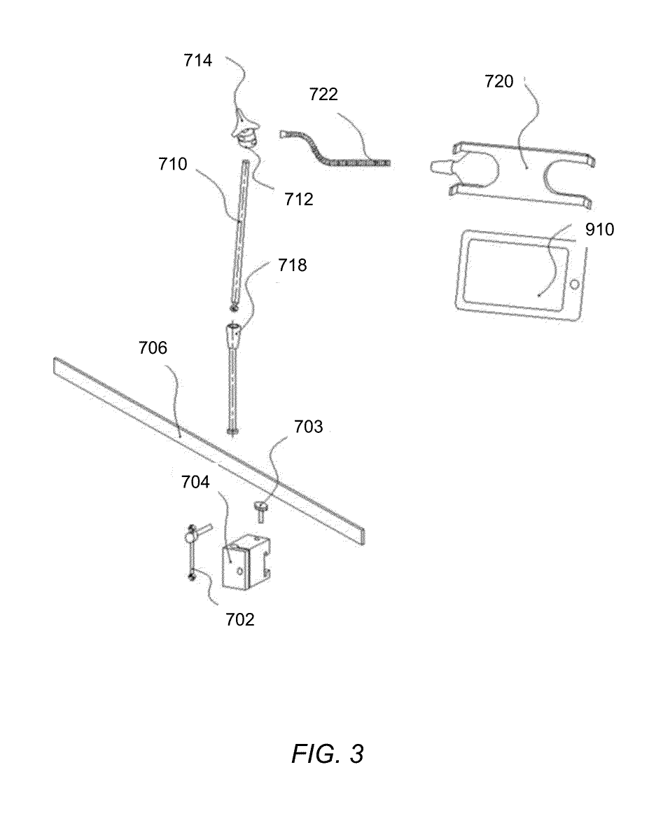

FIG. 3 is an exploded view of a system 700 having a screen holder 720, which can be connected rigidly or by ball joint. The moveable screen holder system 700 includes a handle 702, a support 704, and a flat ruler 706. A screw 703 can be provided for securing a position of the support 704 on the flat ruler 706. The system 700 includes a ball joint 718 coupling the support 704 to a second lever 710. A swivel joint 712, with a fixing handle 714, couples the second lever 710 to a gooseneck 722, which in turn is coupled to a screen holder 720 configured to hold and secure a screen 910 of a display device 900. The system 700 can be coupled to a table 602.

A gooseneck 722 is one way to provide flexibility to allow swiveling and positioning in the desired direction. In the exemplary embodiments illustrated in FIGS. 1, 2A, 2B, 2C, 2D, and 2G, the levers 710, 716 are adjusted by the handle 814 so as to move the positioning apparatus up or down vertically. The ruler 806 will allow movement for the device for horizontal positioning. Additional means of movement can include swivel joints 712. The swivel joints 712 can either be in the intermediate of the device 700 or at the point which adjoins the screen 910 to the system 700.

As shown in FIG. 2C, a screen holder 720 is provide that will allow for the maximum resistance of force during operating. One possible drawback is that this arrangement will not allow for the placement of a semi-transparent screen to see the real ambience of the incision area.

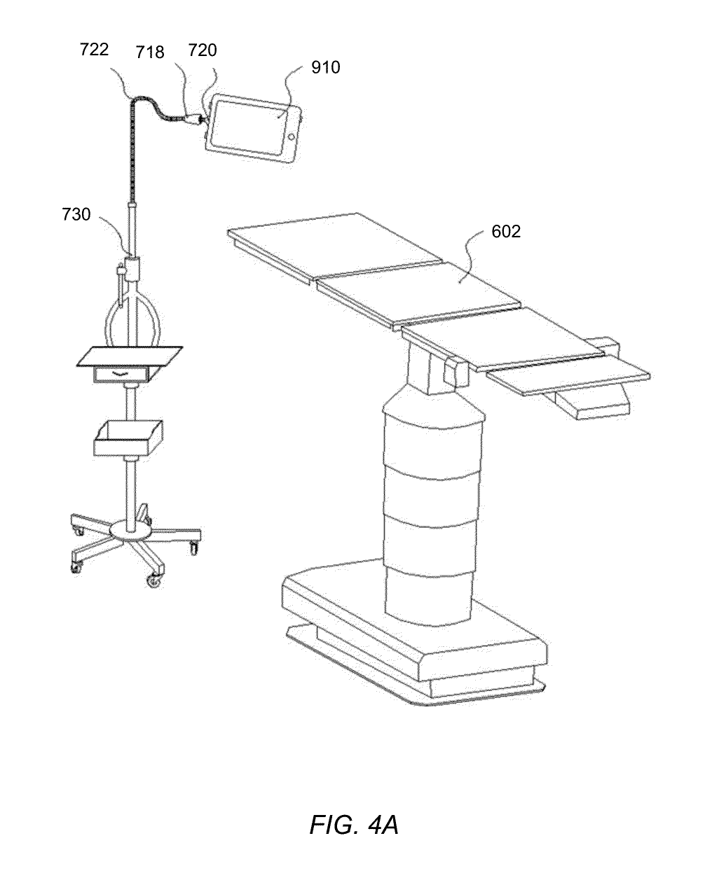

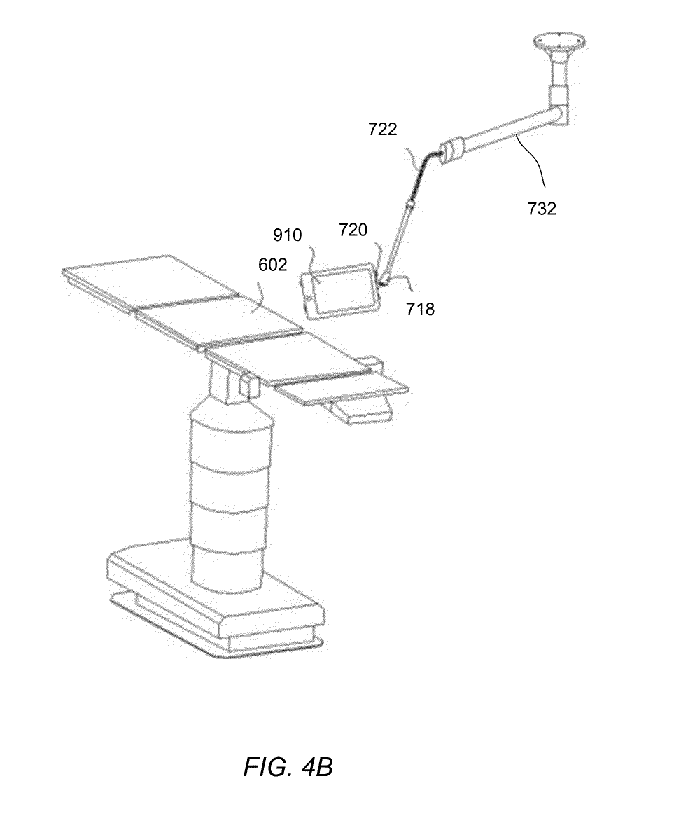

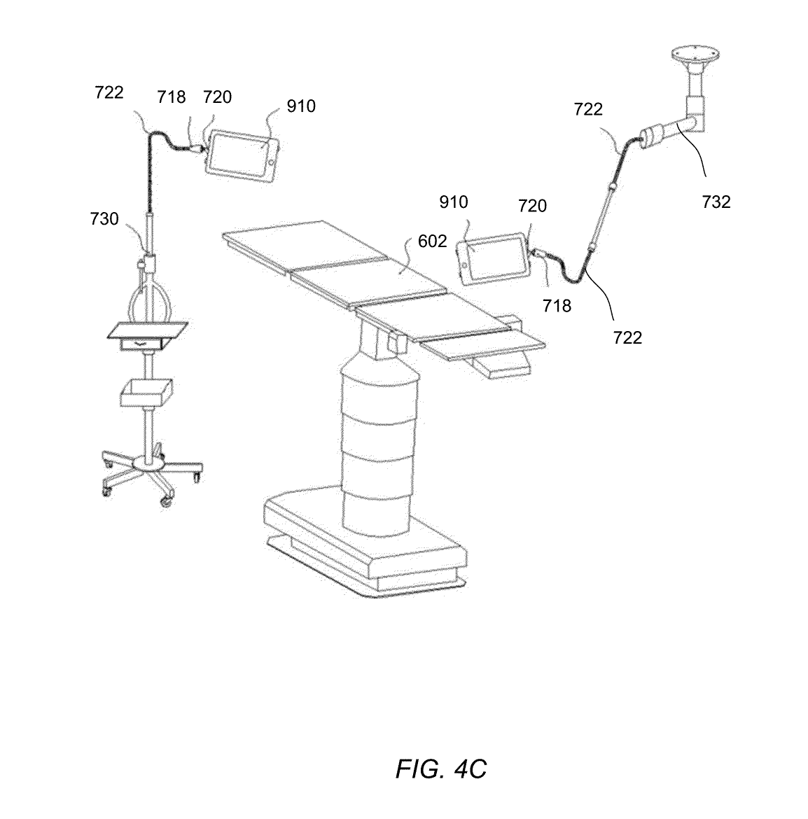

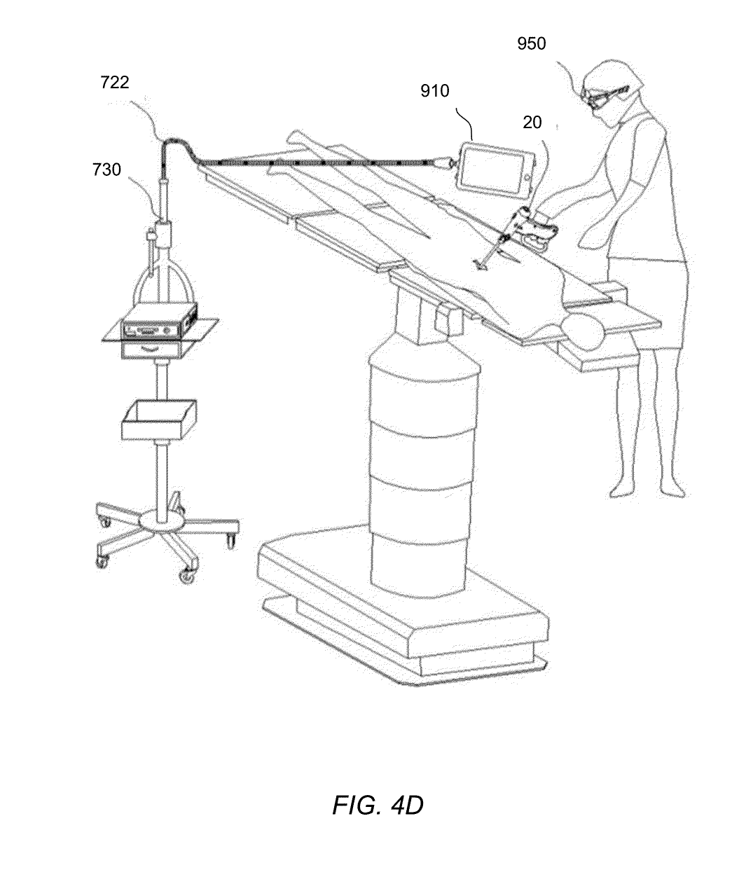

Therefore, in alternative embodiments, the screen 910 can also be attached only to a lateral side of screen 910 as shown for example in FIGS. 2A, 2E, and 2G. One of ordinary skill in the art will understand that this arrangement will allow for the surgeon to see both the streaming images from the endoscope as well as the ambient view of the patient hence maximizing hand eye coordination and improving the probability of success in a surgical procedure. The exemplary embodiments illustrated in FIGS. 1 and 2A-2G are not limited to a bed 602 attachment. With reference to FIGS. 4A-4D, the exemplary features can be on, for example, a stand 730 that can be fixed or moveable with wheels, a side wall mount (not shown), and/or a ceiling mount 732. FIG. 4A illustrates an example of a screen 910 on a stand 730 having wheels. This arrangement can include (a) shelf(s) to put a light source and drawers that can store other medical supplies and a handle that will allow it to be easily moved around. FIG. 4B illustrates an example of a ceiling mount 732 with a gooseneck 722 in the intermediate of the arm of the system 700. FIG. 4C illustrates an example of a plurality of screens 910 that can be used together. In this arrangement, the ceiling mount 732 has both a gooseneck 722 at the distal and intermediate portion. FIG. 4D is an illustration of a doctor using the system 700 and display 900 shown in the embodiment of FIG. 4A.

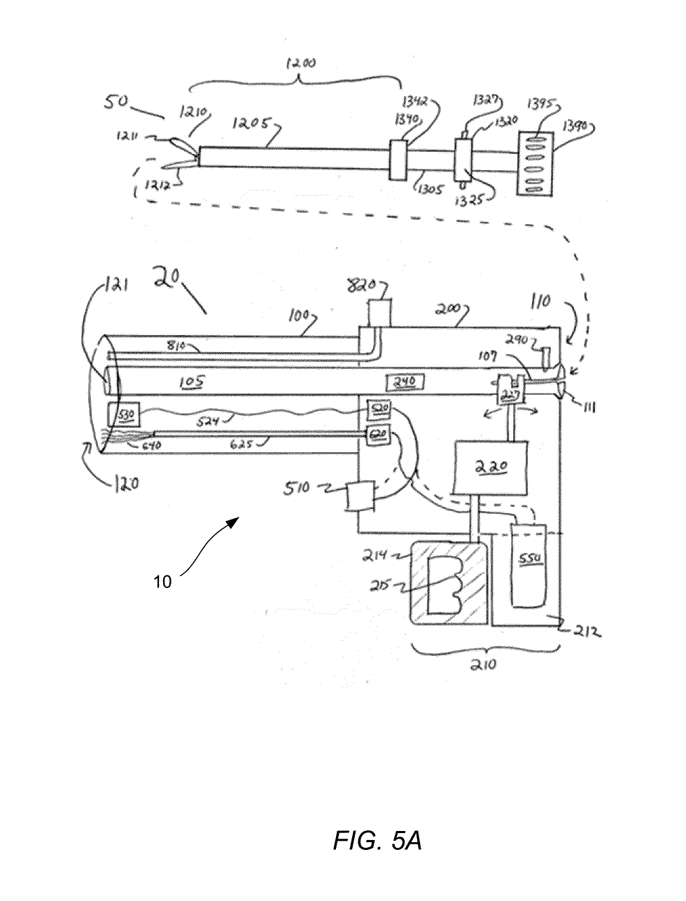

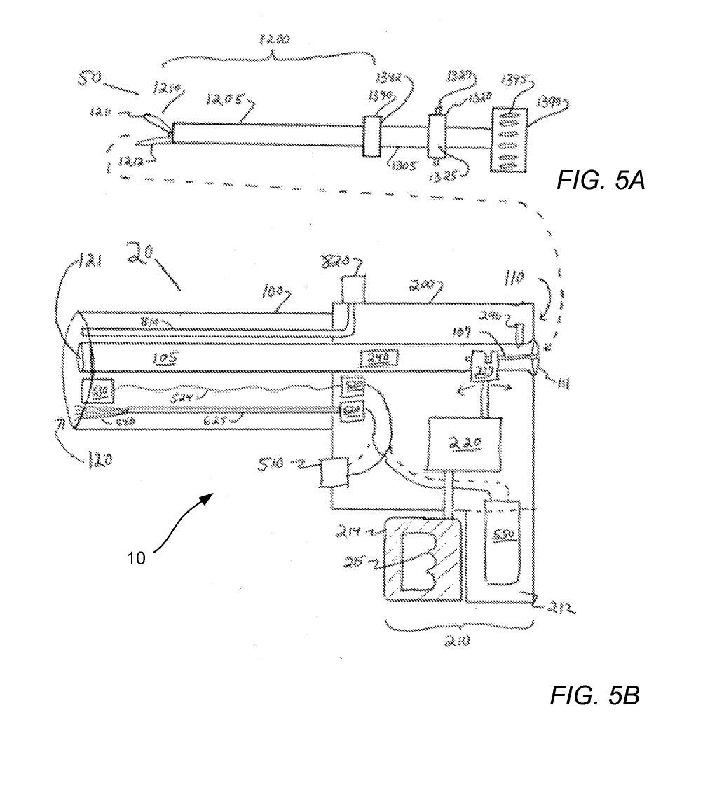

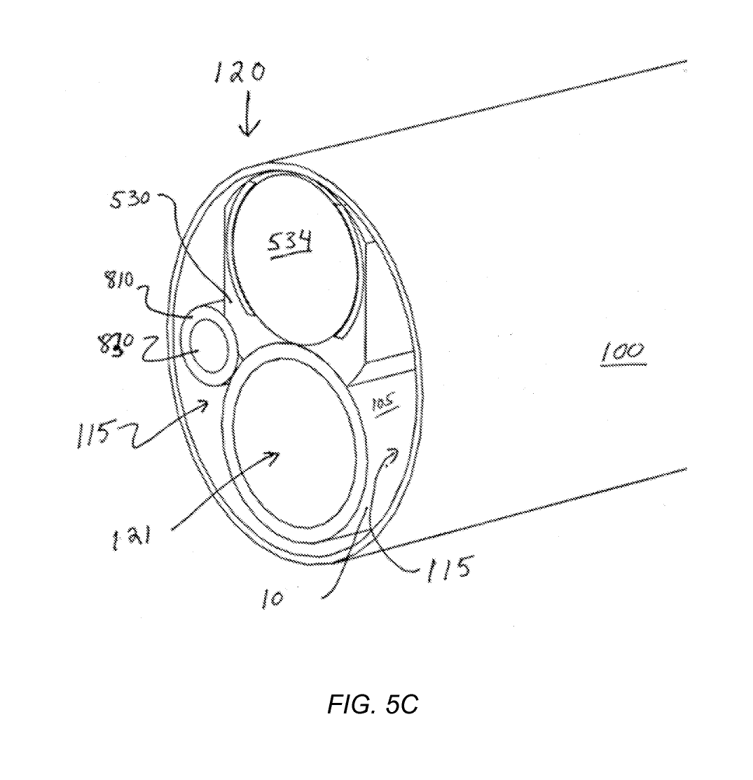

FIGS. 5A and 5B illustrate a functional block diagram schematically illustrating elements of an exemplary endoscopic surgical system 10. The system 10 can include, for example, an endoscopic surgical tool 20 and at least one surgical instrument 50. The illustrated surgical tool 20 comprises a generally tubular body assembly 100 and a control handle assembly 200. The body assembly 100 comprises a proximal end 110 and a distal end 120 and generally penetrates the handle assembly 200, wherein the handle assembly 200 is substantially disposed at a proximal end 110. The surgical instrument 50 is designed to be irreplaceably inserted into the body assembly 100 through an insertion port 111 at the proximal end 110, as will be discussed below. In use, a surgeon inserts or has an assistant insert an instrument 50 into body assembly 100, holds the system 10 by a handle grip 210 and inserts a distal end 120 into a patient through a pre-prepared surgical slit that may be conventionally held open.

The tubular body assembly 100 comprises an instrument guide channel 105. The guide channel 105 comprises a hollow tube that traverses at least the entire length of body assembly 100, starting at the insertion port 111 at the proximal end 110 and ending at an exit port 121 at the distal end 120. As will be described below in the context of an exemplary embodiment, the guide channel 105 typically may comprise multiple, concatenated segments of sequentially decreasing diameter. Additionally, the particular segments may be designed to facilitate mechanical interactions between the surgical tool 20 and the surgical instrument 50. In particular, the channel guide 105 comprises one or two open-ended drive-pin guide slots 107 disposed to run axially, starting with their open end at proximal end 110 and ending at a pre-determined distance towards the distal end 120. The guide slots' end points can be a design choice. Typically, the slots are disposed diametrically opposed in the X-Y plane as illustrated in the FIGS. 5A-5C. In other embodiments, the slot or slots may be disposed elsewhere around the circumference of the channel guide 105.

The surgical tool 20 further comprises a video imaging subsystem 500, the disparate elements of the subsystem being disposed at least partly in the body assembly 100. The imaging subsystem 500 comprises two major elements: a miniaturized video camera head 530 that contains an image sensor and various image forming optical elements disposed at distal end 120 and a video processor 520, disposed conveniently within the system 10. The video processor 520 is configured to work co-operatively and conventionally with the electronics in the camera head 530 to produce a standard format video stream.

Some elements of imaging subsystem 500 may be disposed in the handle assembly 200 when it is convenient to the designer to do so. The power supplies (for example, batteries) and the human interface control devices are typically disposed in the handle assembly 200. Alternatively, the power supplies and/or control devices may be located remotely from the system 10, in which configuration a suitable electrical connector is provided.

In one exemplary embodiment, the video imaging subsystem 500 comprises a display signal output interface 510. The output interface 510 is designed to provide the connection between the tubular body assembly 100 or the handle assembly 200 and an auxiliary video display device (not illustrated). The output interface 510 may be wired or wireless, may comprise a signal only interface or a signal plus power interface, and may be electronic, mechanical, or both. In some exemplary embodiments, the auxiliary video display device may comprise a video processor 520. In one exemplary embodiment, the auxiliary video display device is both mechanically attached to the system 10 at the output interface 510 and receives a video image signal from the imaging subsystem 500 through the output interface 510. In other exemplary embodiments, the output interface 510 may also be used to connect the system's electrical components to an external power source. That is, the output interface 510 may be used as the above mentioned suitable electrical connector.

The surgical tool 20 further comprises an illumination subsystem, the disparate elements of the subsystem being disposed in the body assembly 100 and the handle assembly 200. The illumination subsystem can comprise, for example, two major elements; a low power light source assembly 620, which contains, typically, a low-power white light source such as a “white” LED and miscellaneous coupling optics, if needed, and an incoherent bundle 645 of multi-mode optical fibers. The fiber bundle 645 delivers light from the conveniently disposed source assembly 620 to the distal end 120. The fiber bundle 645 may be encased in a protective jacket for part of the distance between the source assembly 620 and the distal end 120 may be totally unjacketed. At the distal end 120, the fibers in the bundle are spread out to fill the empty spaces around the other elements, for example, the video head 530 and the exit port 121, disposed at the distal end 120. The electrical power and/or the control devices for the light source may be provided by a battery, typically located in the handle assembly 200, or they may be located remotely from the system 10, in which configuration a suitable electrical connector is provided. Note that the power supplies and the control devices may be shared between the video imaging subsystem 500 and the illumination subsystem 600.

In some exemplary embodiments, the surgical tool 20 further comprises an irrigation channel 800. The irrigation channel 800 can include a conventional design and typically comprises a fluid input/output (I/O) port 820 disposed very generally towards proximal end 110 and a fluid transport tube 810 running through the interior of body assembly 100 from I/O port 820 to an irrigation port 830 disposed at the distal end 120.

In some exemplary embodiments, one or more human interface control devices 550 for the imaging subsystem 500 are incorporated into the handle grip 210. Disposition of the control devices 550 (for example, switches to control on/off, brightness, contrast, etc.) as part of the handle grip 210 allows the surgeon to adjust the imaging system performance to meet his needs without releasing the grip 210 or releasing the instrument in his other hand or calling instructions to an assistant.

FIG. 5C is a detailed illustration of the distal end of tubular body assembly 100. The open aperture distal end 121 of guide channel 105 is generally flush with the end of tubular body assembly 100 as is the irrigation port 820 of the fluid transport tube 810. The video camera head 530 is also disposed at the distal end of body assembly 100. In this view, a lens 534 is disposed to look out toward the surgical field beyond the distal end 120. Generally, the lens 534 is set back slightly from being flush with the end of the tubular body assembly 100 for self-protection and cleanliness. Although not illustrated, the output tips of the optical fibers carrying illumination from a source in the handle assembly 200 are disposed in otherwise vacant spaces 115 at the distal end 120.

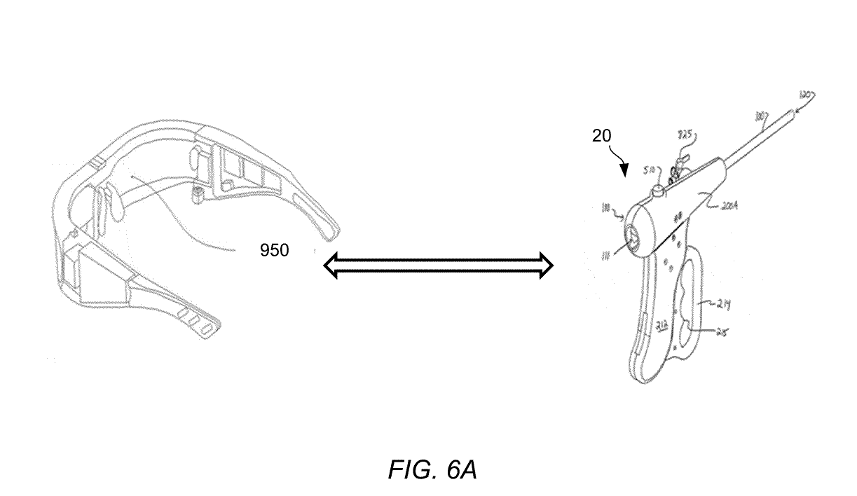

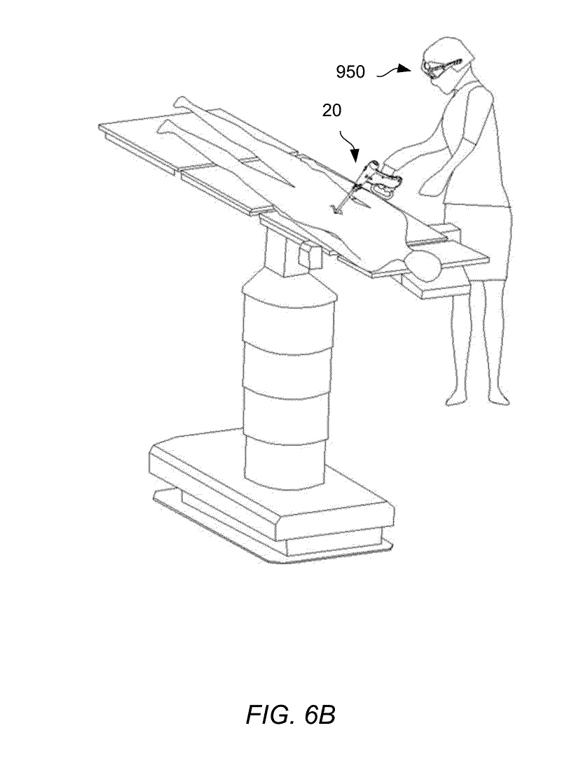

FIGS. 6A and 6B illustrate optical head mounted display glasses 950 configured to be in electronic communication with an endoscope 20. The illustrated glasses 950 can be conventional glasses available, for example, from Osterhout Group, as described for example in U.S. Pat. No. 8,467,133 and U.S. Pat. No. 8,477,425, each of which is incorporated herein by reference in their entirety. The glasses 950 are an eye glass with a semi transparent display the augmented reality optical elements that are embedded in the arm portions of the eyepiece. Images may be projected with a projector and can projected onto both lenses, or alternatively, projected onto a single lens. The region that is in directly in front of the endoscope is transmitted to the optical head mount from the endoscope lens 530. In this case, a surgeon uses the glasses 950 as a standalone with no additional screens; however, a surgeon can use this arrangement in conjunction with other monitors or screens 910. In the illustrated embodiment, a doctor is using a rigid endoscopic device, while in other situations a doctor can use a flexible endoscopic device.

The glasses 950 can be in direct electronic communication with the endoscope 20 with wires or wireless through a microprocessor that is connected with the imaging module through interface 510. The medical professional can view the ambience proximate to the incision sight/opening as well frame stream from the endoscope 20 due to a semi-transparent nature of the display module of the glasses 950. The medical professional can control the functions, for example, through voice command and/or a touch or non-touch keyboard. These glasses 950 will enable a surgeon to see both the streaming images as well as the ambient view of the incision\opening area hence enabling better and eye coordination.

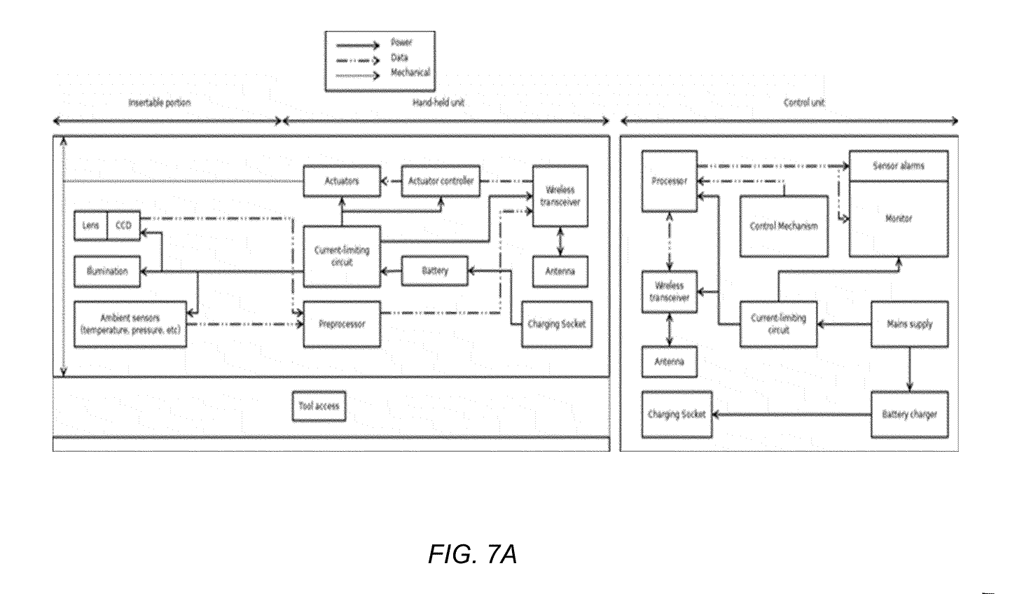

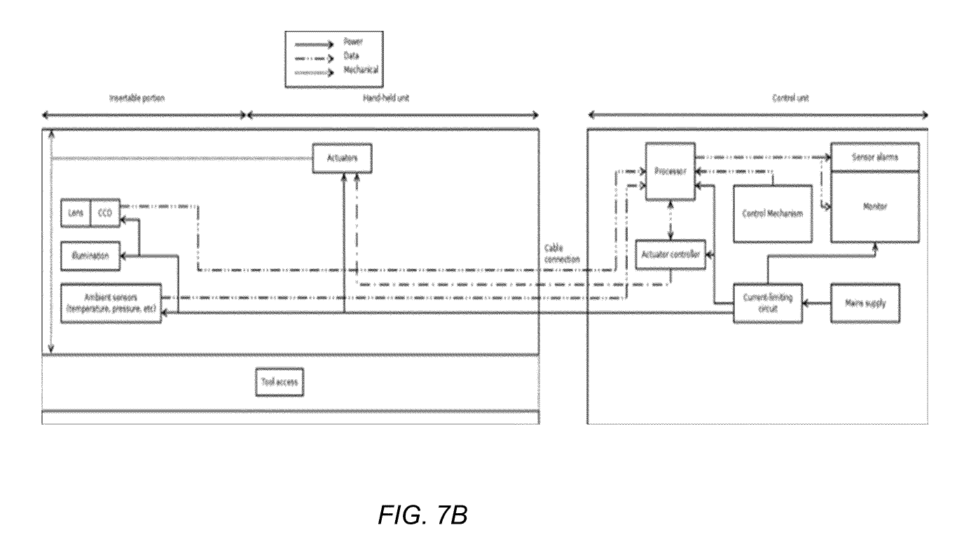

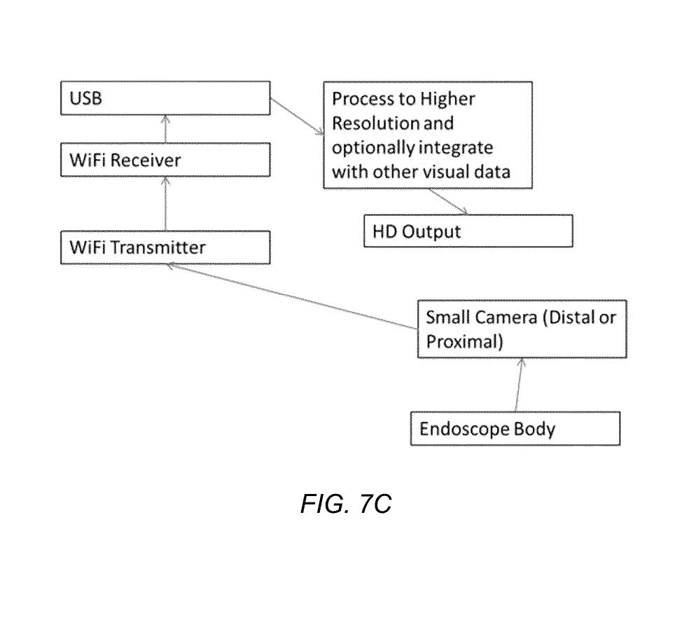

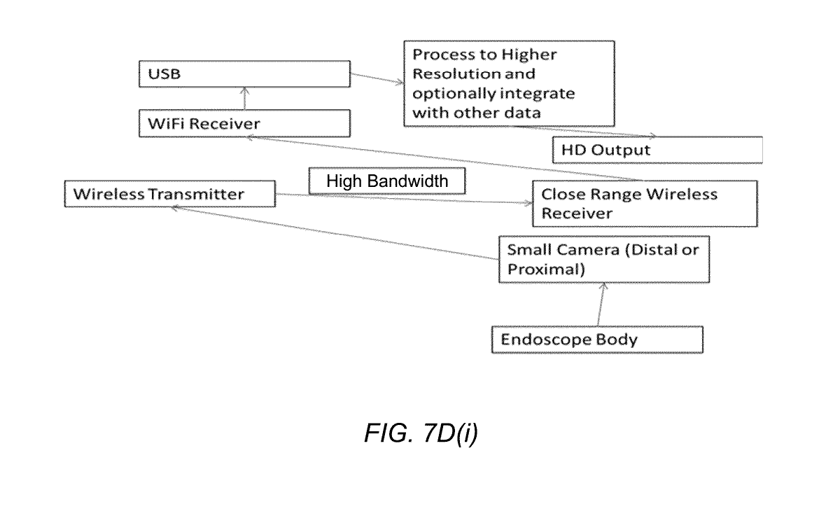

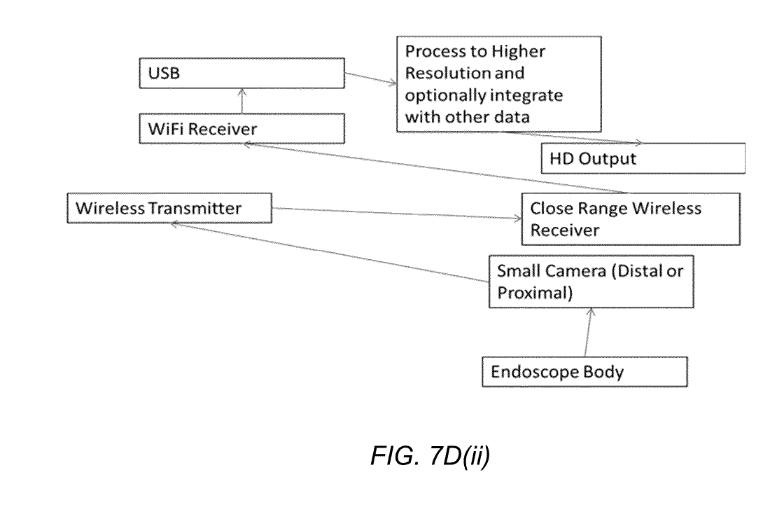

FIG. 7A schematically illustrates a flowchart showing components and operation of an exemplary wireless endoscope 20 with system 700 and display 900. FIG. 7B illustrates a flowchart showing components and operation of an exemplary wired endoscope 20 with system 700 and display 900. FIGS. 7C and 7D schematically illustrate alternative exemplary embodiments of the wireless system (e.g., wireless endoscope 20 with system 700 and display 900). In each of these embodiments, an image directly in front of the endoscope 20 is relayed to the viewing apparatus or display 900.

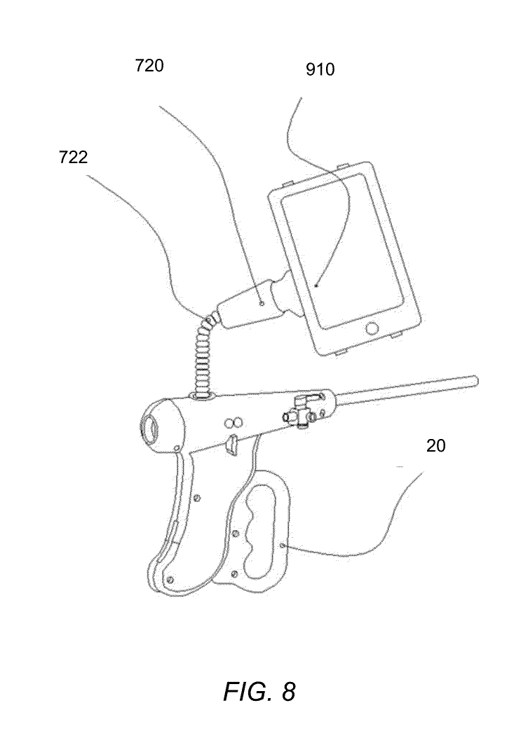

FIG. 8 illustrates an exemplary embodiment in which a viewing apparatus or display 900 is attached to the endoscope 20 by a gooseneck 722 which will allow the surgeon to move the screen 910 to the desired position. The screen 910 can also be moveable and attached by the means and methods described and shown, for example, in FIGS. 2A-4D.

An exemplary embodiment is directed to an endoscope video imaging system comprising an endoscope 20 having an image sensor; a plurality of image forming optical elements; a video processor; a video processor display signal output interface; an illumination subsystem with one or more light sources; and at least one light transfer and projection element, wherein the endoscope 20 is in communication and sends images through an electronic interface to a head-mounted eyepiece 850 configured to be worn by a user, wherein the eyepiece 950 includes an optical assembly combined with displayed content, an integrated processor for handling content for display to the user, and an integrated image source for introducing the content from the camera to the optical assembly.

An exemplary embodiment of the invention comprises a video endoscope 20 that is in electronic communication with a head piece 950, wherein a medical worker views the real ambience proximate to the incision sight as well as the augmented frame stream on a semi-transparent display module streamed from the endoscope 20.

An exemplary embodiment is directed to a head-mounted eyepiece 950 in communication with an endoscope 20, wherein the eyepiece 950 includes an optical assembly, an integrated processor for handling content for display to the user, and an integrated image source for introducing the content to the optical assembly, whereby the lens is substantially transparent to enable a medical worker to be in simultaneous visual communication with both the incision area\opening area of the patient and image stream from the endoscope 20.

The endoscope 20 can be flexible or the endoscope can be rigid. The endoscope 20 is configured to accept surgical tools.

An exemplary embodiment is directed to an endoscope video imaging system comprising an endoscope 20 having an image sensor, image forming optical elements, a video processor, a video processor display signal output interface, an illumination subsystem, one or more light sources, at least one light transfer and projection element; and an image display 900 to be in communication and receive images from the endoscope 20, the image display 900 is coupled to a moveable assembly system 700, the moveable assembly system 700 providing at least two degrees of freedom of movement, wherein the position of the screen 910 of the display 900 can be manipulated into a position that is proximate for viewing of both the ambient view of the patient and the image display 900, and wherein the screen 910 is in an intermediate area in-between the physicians or medical workers line of sight and the opening area for the endoscope 20.

The screen 910 can be a semitransparent display screen, wherein the user views the display screen 910 combined with displayed content, an integrated processor for handling content for display to the user, and an integrated image source for introducing the content to the assembly.

The screen 910 can include a cover that is made of a material that is one of autoclaveable and disposable. The imaging module can include an angle variation from 0-180 degrees in X-axis and 0-180 degrees in a Y-axis. The screen movement can be configured to be oriented in a substantially horizontal manner over a patient. The screen movement can be at least one of angular and rotational along at-least one axial plane. The screen 910 can be movable through an actuator between a first position and a second position.

In an embodiment, the system 700 further comprises a strut and crank configured to provide screen movement. In an embodiment, the system 700 further comprises a gooseneck 722 configured to provide screen movement. In an embodiment, the system 700 further comprises a piston and spring configured to provide screen movement. In an embodiment, the system 700 further comprises a cable having a fixed path length configured to provide screen movement. In an embodiment, the system 700 further comprises a series of joints configured to provide screen movement. In an embodiment, the system 700 further comprises a swivel joint 712 configured to provide screen movement. In an embodiment, the system 700 further comprises a spring which is attached to its bearing configured to provide screen movement. In an embodiment, the system 700 further comprises a ball-and-socket joint 718 configured to provide screen movement. In an embodiment, the screen 910 is held by a hinge 724. The screen 910 can move through folding arm movement.

In an embodiment, a surface area of the screen 910 is 15-75 square inches. In an embodiment, a surface area of the screen 910 is 75-130 square inches. In an embodiment, a surface area of the screen 910 is 130-180 square inches. In an embodiment, a surface area of the screen 910 is 180-250 square inches.

In an embodiment, the screen 910 is configured to be up to 10 inches from an incision sight. In an embodiment, the screen 910 is configured to be up to 10-20 inches from an incision sight. In an embodiment, the screen 910 is configured to be up to 20-30 inches from an incision sight. In an embodiment, the screen 910 is configured to be up to 10 inches from a surgeon's eyes. In an embodiment, the screen 910 is configured to be up to 10-20 inches from a surgeon's eyes. In an embodiment, the screen 910 is configured to be up to 20-30 inches from a surgeon's eyes.

In an embodiment, an attachment is provided that adjoins an adjuster apparatus to the screen 910 is connected to a side wall mount. In an embodiment, the screen 910 is attached to a stand 730. In an embodiment, the stand has wheels. In an embodiment, the apparatus is attached to a ceiling mount 732.

In an embodiment, a method of manufacturing the system and a method of using the system are provided. The method of manufacturing the system 700 includes providing a moveable screen holder system 700, as shown in any one of FIGS. 1-8.

The present invention has been described herein in terms of several preferred embodiments. However, modifications and additions to these embodiments will become apparent to those of ordinary skill in the art upon a reading of the foregoing description. It is intended that all such modifications and additions comprise a part of the present invention to the extent that they fall within the scope of the several claims appended hereto.

We claim:

1. An endoscope video imaging system comprising:
an image sensor;
a plurality of image forming optical elements;
a video processor;
a video processor display signal output interface;
an illumination subsystem with one or more light sources; and
at least one light transfer and projection element,
wherein the endoscope is in communication and sends images through an electronic interface to a head-mounted eyepiece configured to be worn by a user, wherein the eyepiece includes an optical assembly combined with displayed content, an integrated processor for handling content for display to the user, and an integrated image source for introducing the content from the camera to the optical assembly.

2. A head-mounted eyepiece in communication with an endoscope, wherein the eyepiece includes an optical assembly, an integrated processor for handling content for display to the user, and an integrated image source for introducing the content to the optical assembly, whereby the lens is substantially transparent to enable a medical worker to be in simultaneous visual communication with both the incision area\opening area of the patient and image stream from the endoscope.

3. The system according to claim 1, wherein the endoscope is flexible.

4. The system according to claim 1, wherein the endoscope is rigid.

5. The system according to claim 1, wherein the endoscope is configured to accept surgical tools.

6. An endoscope video imaging system comprising:
an image sensor,
image forming optical elements,
a video processor,
a video processor display signal output interface,
an illumination subsystem,
one or more light sources,
at least one light transfer and projection element; and
an image display to be in communication and receive images from the endoscope,
the image display device is coupled to a moveable assembly, the moveable assembly providing at least two degrees of freedom of movement,
wherein the position of the screen can be manipulated into a position that is proximate for viewing of both the ambient view of the patient and the image display device, and
wherein the screen is in an intermediate area in-between the physicians or medical workers line of sight and the opening area for the endoscopic device.

7. The system according to claim 6, wherein the screen is a semitransparent display screen,
wherein the user views the display screen combined with displayed content, an integrated processor for handling content for display to the user, and an integrated image source for introducing the content to the assembly.

8. The system according to claim 6, wherein the screen has a cover that is made of a material that is one of autoclaveable and disposable.

9. The system according to claim 6, wherein the imaging module comprises an angle variation from 0-180 degrees in X-axis and 0-180 degrees in a Y-axis.

10. The system according to claim 6, wherein the endoscope is flexible.

11. The system according to claim 6, wherein the endoscope is rigid and configured to accept surgical tools.

12. The system according to claim 6, wherein the screen movement is configured to be oriented in a substantially horizontal manner over a patient.

14. The system according to claim 6, wherein the screen movement is at least one of angular and rotational along at-least one axial plane.

15. The system according to claim 6, wherein the screen is movable through an actuator between a first position and a second position.

16. The system according to claim 6, further comprising:
a strut and crank configured to provide screen movement.

17. The system according to claim 6, further comprising:
a gooseneck configured to provide screen movement.

18. The system according to claim 6, further comprising:
a piston and spring configured to provide screen movement.

19. The system according to claim 6, further comprising:
a cable having a fixed path length configured to provide screen movement.

20. The system according to claim 6, further comprising:
a series of joints configured to provide screen movement.

21. The system according to claim 6, further comprising:
a swivel joint configured to provide screen movement.

22. The system according to claim 6, further comprising:
a spring which is attached to its bearing configured to provide screen movement.

23. The system according to claim 6, further comprising:
a ball-and-socket configured to provide screen movement.

24. The system according to claim 6, wherein the screen is held by a hinge.

25. The system according to claim 6, wherein the screen moves through folding arm movement.

26. The system according to claim 6, wherein a surface area of the screen is 15-75 square inches.

27. The system according to claim 6, wherein a surface area of the screen is 75-130 square inches.

28. The system according to claim 6, wherein a surface area of the screen is 130-180 square inches.

29. The system according to claim 6, wherein a surface area of the screen is 180-250 square inches.

30. The system according to claim 6, wherein the screen is configured to be up to 10 inches from a incision sight.

31. The system according to claim 6, wherein the screen is configured to be up to 10-20 inches from an incision sight.

32. The system according to claim 6, wherein the screen is configured to be up to 20-30 inches from an incision sight.

30. The system according to claim 6, wherein the screen is configured to be up to 10 inches from a surgeon's eyes.

31. The system according to claim 6, wherein the screen is configured to be up to 10-20 inches from a surgeon's eyes.

32. The system according to claim 6, wherein the screen is configured to be up to 20-30 inches from a surgeon's eyes.

33. The system according to claim 6, wherein an attachment that adjoins an adjuster apparatus to the screen is connected to a side wall of the screen.

34. The system according to claim 6, wherein the screen is attached to a stand.

35. The system according to claim 34, wherein the stand has wheels.

36. The system according to claim 6, wherein the apparatus is attached to a ceiling mount.

37. A method of manufacturing the system of claim 1.

38. A method of using the system of claim 1.
